(19) United States
(12) Patent Application Publication
(10) Pub. No.: US 2004/0028676 A1
(43) Pub. Date: Feb. 12, 2004

(54) Swallowing system tissue modifier

(76) Inventors: Dean A. Klein, North Oaks, MN (US); James D. Brazil, Braham, MN (US)

Correspondence Address: FAEGRE & BENSON LLP, 2200 WELLS FARGO CENTER, 90 SOUTH 7TH STREET, MINNEAPOLIS, MN, 55402, US

(21) Appl. No.: 10/212,837

(22) Filed: Aug. 6, 2002

(51) Int. Cl. A61K 33/44

(52) U.S. Cl. 424/125

(57) ABSTRACT

The present invention provides a method for treating disorders of the swallowing system, including injecting a modifier composed of biocompatible particles and a biocompatible carrier into one or more desired tissue sites. The present invention may be injected at a tissue site of a patient's mouth, pharynx, nasal passages, esophagus, trachea or other suitable tissue sites to treat snoring and other tissue disorders.

### BACKGROUND

The swallowing system is composed of a single tube which widens in an upper region to form a plurality of cavities. The tube divides at a lower end into a feeding tube (i.e. the esophagus) and a breathing tube (i.e. the trachea). Each cavity, formed by the enlargement of a portion of the tube, serves initially either the function of feeding (i.e. oral cavity) or breathing (i.e. nasal passages).

The feeding and respiratory systems share a portion of the pharynx between the area behind the tongue and the area at the entrance to the larynx and the entrance to the esophagus. These systems also may share the oral cavity when a person breathes through his or her mouth.

Because these two systems, collectively referred to herein as the “swallowing system,” share portions of the same tube, valves are used to support the desired activity and to separate the two systems. For example, during breathing, certain valves open to allow air to enter the nose, larynx and trachea, and close to prevent air from entering the esophagus and lower digestive tract. During swallowing, valves move food into the digestive system while preventing movement into the respiratory system.

Swallowing system disorders are common conditions in people. One of the most prevalent swallowing system disorders is snoring. It is estimated that 90 million Americans over the age of eighteen snore. Although not normally considered life threatening, habitual snoring may have an adverse affect on a person suffering from snoring, as well as persons that sleep in the vicinity of the snorer.

Snoring is generally caused by dynamic response of soft tissue in the swallowing system to the passage of air through the swallowing system. As air flows past the soft tissue site, the soft tissue vibrates and/or flaps against adjacent tissue causing an audible “snoring” noise. The soft tissue sites in the swallowing system that may contribute to snoring include the soft palate, nasal passages, tongue, pharynx 30 and other tissue sites that vibrate in response to the passage of air.

One example of a soft tissue site that is a common source of snoring is the soft palate. One end of the soft palate is secured to the hard palate in the pharynx. A second end of the soft palate is not attached or supported by bony tissue so that it is free to move. During normal breathing, the soft palate simply hangs from the hard palate. During swallowing, the soft palate closes off the nasal passages to prevent food or drink from passing into the nose. However, during sleep, the soft palate may respond to the passage of air by rapidly opening and closing the nasal passages to create the snoring noise.

Although snoring itself is not considered life threatening, it is rarely associated with a more dangerous disorder called sleep apnea. Sleep apnea is characterized by periodic cessation of breathing during sleep. Sleep apnea may be caused by closure of the throat during sleep.

There are numerous treatments for snoring, including pharmaceutical compositions, dental appliances and surgery. U.S. Pat. No. 6,250,307 (Conrad), reports several surgical procedures for treating snoring. In a first treatment called uvulopalatopharyngoplasty, a patient's soft palate is surgically reduced to lessen the dynamic response of the soft palate to the flow of air. A second surgical method involves laser scarring the soft palate to alter its dynamic response. Both of these methods have several drawbacks. First, both involve an invasive procedure resulting in significant trauma and discomfort. Furthermore, if too much of the soft palate is altered, the ability of the soft palate to close during swallowing may be permanently compromised. Further yet, the treatment is only reported for use on the soft palate, while snoring may be caused by dynamic movement of soft tissues of the tongue, throat and nasal passages as well.

Conrad also reports a device that may be implanted into the soft palate through a surgical incision. The implant may come in several shapes, including beads of about 2-4 millimeters in diameter, a stiffening strip and a liquid filled bladder. Conrad further reports that these implants treat snoring by altering the mass, rigidity and/or shape of the soft palate to affect the dynamic response of the soft palate to the flow of air. Although the device reported in Conrad overcomes some of the problems associated with the aforementioned surgical methods, it still has several shortcomings. First, the implant requires an incision to be made in the soft palate. Second, because the implants are large relative to the dimensions of the soft palate, implantation may be relatively imprecise and may have a tendency to alter the dynamic response of the soft tissue more than is minimally necessary to prevent snoring, possibly causing the soft palate to lose functionality. Third, Conrad merely reports implantation in the soft palate. Snoring may be caused by dynamic movement of soft tissues other than the soft palate, for example the nasal passages, tongue and/or pharynx.

Additionally, none of the procedures discussed in Conrad are reported for use in treating a variety of other tissue conditions of the swallowing system, including stagnant pockets, sleep apnea, soft tissue cavities caused by the removal of cysts or tumors, speech impediments, difficulty swallowing, voice conditions, aspiration and jaw disorders.

### SUMMARY OF THE INVENTION

The present invention provides a swallowing system tissue modifier that may be injected into various tissue sites of the swallowing system of a patient to treat a variety of disorders and/or conditions. As used herein, the term “swallowing system” refers collectively to the feeding and respiratory systems, generally commencing in the oral and nasal passages and terminating in the vicinity of the upper esophageal and tracheal regions.

In one embodiment, the present invention provides a method for modifying the tissue of the swallowing system of a patient. A tissue modifier composed of biocompatible particles suspended in a biocompatible carrier is injected at a tissue site of a patient's swallowing system. The tissue site may include the tongue, lips, soft palate, pharynx, nasal passages, true vocal cords, false vocal cords, epiglottis, trachea, cricoid cartilage and/or other suitable tissue sites of the swallowing system. The modifier may relief conditions or disorders such as snoring, stagnant pockets, sleep apnea, soft tissue cavities, speech impediments, swallowing disorders, aspiration, voice conditions and other conditions affected by or interacting with the shape and/or structure of the tissue of the swallowing system.

Suitable particles used in accordance with this embodiment are biocompatible and/or inert and injectable. In one example, the biocompatible particles are ceramic, polymeric or carbon particles. In another example, the biocompatible particles are carbon coated particulate substrates. In a further example, the biocompatible particles include isotropic pyrolytic carbon coated graphite or polymeric particulate substrates.

The biocompatible carrier of this embodiment may be any biologically compatible material capable of delivering the particles to a desired tissue site. The carrier may be a solution, liquid, gel, suspension or slurry (collectively referred to herein as a “solution”) including, for example, solutions containing β-glucan, collagen and/or saline. The combination of the biocompatible particles and the carrier provide a modifier for injection into a tissue site, and may have a viscosity of between about 10 and 75,000 centipoise. Embodiments of the present invention may be delivered to the subcutaneous, mucosal, submucosal or muscular layer of the desired tissue site by injection using a hypodermic needle and syringe, or another similar instrument.

In another embodiment, the present invention provides a method of modifying the tissue of the swallowing system wherein the modifier is composed of an injectable biocompatible solution that is free of biocompatible particles. The solution may include, for example, β-glucan or collagen. In this embodiment, certain biocompatible solutions may be used to temporarily modify the desired tissue site. If desired, a modifier composed of biocompatible particles and a biocompatible carrier may then be subsequently injected into the same site for substantially permanent modification. Alternatively, certain biocompatible solutions, such as solutions containing β-glucan, may produce a fibroid response in the tissue site. In these embodiments, permanent modification is achieved by injecting the biocompatible solution free of particles into the desired tissue site.

In yet another embodiment, the present invention provides a method for treating snoring by modifying tissue sites of the swallowing system. The modifier includes biocompatible particles combined with a biocompatible carrier that is injected into a soft tissue site of the patient to affect the dynamic response of the soft tissue to the passage of air while the patient breathes. The modifier may alter the mass, rigidity and/or geometry of the tissue site to affect its dynamic response.

The present invention possesses performance characteristics not apparent with other swallowing system modifiers. For example, unlike reported modifiers, the present invention allows for precise injection into the desired tissue site. Additionally, the amount of modifier used may be carefully controlled, reducing the risk of adversely affecting the normal functions of the soft tissue. Further, the present invention may be injected into any suitable tissue site of the swallowing system, and is not limited to the soft palate or uvula. Further yet, the present invention is less invasive and traumatic than reported surgical modifications of the tissue of the swallowing system.

### BRIEF DESCRIPTION OF THE DRAWINGS

FIG. 1 illustrates the swallowing system of an adult during normal breathing.

FIG. 2 illustrates the swallowing system of an adult during swallowing.

### DETAILED DESCRIPTION OF THE INVENTION

The present invention provides methods of treating disorders of the swallowing system wherein a tissue modifier is injected into one or more desired tissue sites. The method may be used to treat a variety of conditions, including snoring, sleep apnea, stagnant pockets, soft tissue cavities, speech impediments, aspiration, difficulty swallowing, voice conditions and other conditions caused by tissue characteristics or conditions of the swallowing system.

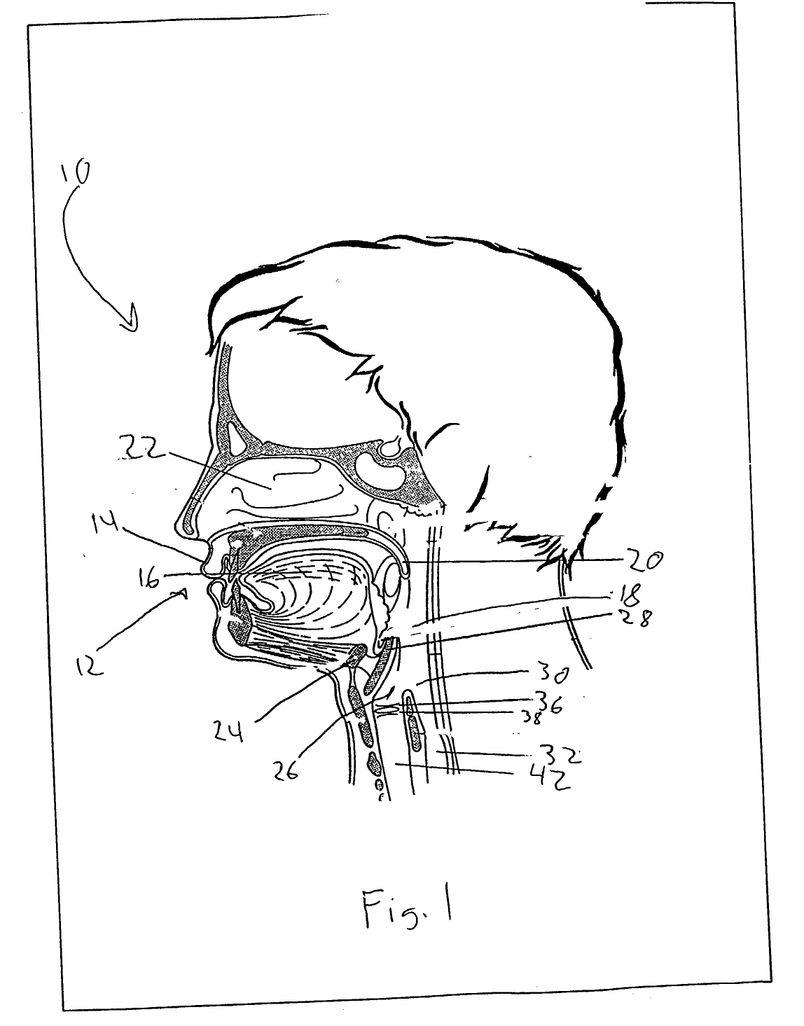

FIG. 1 illustrates the swallowing system 10 of an adult during normal breathing. As previously described, the swallowing system is composed of a series of valves designed to implement one system (i.e. the feeding or respiratory system) and to separate the two systems.

During breathing, one goal of the swallowing system 10 is to move air efficiently into the respiratory system and keep it out of the digestive system. The following events, which may occur chronologically or simultaneously, support this goal. The soft palate 20 relaxes allowing air to enter the nasal passages 22 and pass downward towards the lungs (not shown). The true and false vocal cords 36, 38 remain relaxed and open for the entry of air. The upper esophageal sphincter 30 (“UES”) sustains contraction to close off the top of the esophagus 32 and to prevent air from entering the digestive system.

During swallowing, one goal of the swallowing system 10 is to move food into the digestive system and keep it out of the respiratory system. The following events, which may occur rapidly and/or simultaneously support this goal. Food 44 is propelled from the front to the back of mouth 12 during the oral stage of swallowing. Lips 14 and the side of tongue 16 serve as valves to direct food 44 efficiently toward the pharynx 18. The back of the tongue 16 elevates and moves the food 44 into the pharynx 18 as the pharyngeal swallow is triggered. Muscles in the pharynx 18 contract in a peristaltic wave, moving the food 44 downward. Soft palate 20 elevates to prevent the food 44 from refluxing into the nasal passages 22. Hyoid 24 and larynx 26 elevate, and epiglottis 28 moves downward to protect the entrance to the airway. This movement of the hyoid 24 also initiates relaxation of the UES 30 and opening of esophagus 32. Larynx 26 is pulled forward and upward under the tongue 16, pulling true and false vocal folds 36, 38 together to provide additional airway protection. The food 44 moves through the UES 30 into the esophagus 32. The UES 30 then closes, preventing upward movement of the food 44. Peristaltic movement of the esophagus 18 carries the food 44 to the stomach (not shown).

From the foregoing, it is evident the swallowing system 10 requires coordination of various complex actions to function properly. Malfunction of any one of these actions may cause swallowing system disorders, including snoring, difficulty swallowing, sleep apnea, and aspiration. Further, conditions such as stagnant pockets, speech impediments, tissue cavities, and voice conditions may also be related to the tissues of the swallowing system.

The swallowing system modifier used in certain embodiments of the present invention combines biocompatible particles with a biocompatible carrier. The modifier is preferably capable of injection into a desired swallowing system tissue site.

Almost any suitable biocompatible particle may be used in accordance with the present invention. In one embodiment, the particles are generally made of a durable material, for example, a ceramic, such as zirconium or aluminum, gold, titanium, silver, stainless steel, graphite, isotropic pyrolytic carbon, oxides, polymers, metal alloys and/or combinations thereof. In other embodiments, the particles may be carbon coated particulate substrates. Suitable particulate substrates generally include particles capable of accepting a carbon coating, such as the particulate material described above. The particulate substrates may be carbon coated, for example, with pyrolytic carbon, vitreous carbon, diamond-like carbon or graphite by conventional techniques. Optionally, the particulate substrate may be radiopaque. In one embodiment, the particles include isotropic pyrolytic carbon coated onto a graphite particulate substrate.

The atomic structure of pyrolytic and vitreous carbon is similar to graphite, but the alignment between hexagonal planes of atoms is not as well ordered as in graphite. Pyrolytic carbon is characterized by a more chaotic atomic structure and better bonding between layer planes. The carbon coating provides a generally smooth surface for injection into a tissue site.

Pyrolytic carbon may be produced and coated onto particulate substrate surfaces by known methods. In one technique, hydrocarbons and alloying gases are decomposed to prepare a pyrolytic carbon coating on the particulate substrates. The particulate substrates are contacted with the hydrocarbons and alloying gases in a fluidized or floating bed at a temperature sufficient to cause deposition of pyrolyzed carbon onto the particulate substrate surfaces, typically from about 1200 to 1500°. Inert gas flow is used to float the bed of particulate substrates, optionally including an inert mixing media. The hydrocarbon pyrolysis results in a high carbon, low hydrogen content carbon material being deposited as a solid layer of material onto the particulate substrates.

Alternatively, a carbon coating (sometimes referred to as “ultra-low-temperature isotropic carbon”) may be applied to particulate substrates using any one of other various coating processes for depositing carbon, such as a vacuum vapor deposition process. Such a method uses ion beams generated from any of a variety of known processes, such as the disassociation of CO₂, reactive dissociation in vacuum of a hydrocarbon as a result of a glow discharge, sublimation of a solid graphite source, or cathode sputtering of a graphite source. Gold has been found to be an especially suitable particulate substrate for vacuum vapor deposited carbon. Other particulate substrates, including but not limited to nickel, silver, stainless steel, zirconium, graphite or titanium are also quite acceptable for this type of coating process.

Isotropic carbon may also be applied to temperature-sensitive substrates using physical vapor depositions techniques. Physical vapor deposition involves transferring groups of carbon atoms from a pyrolytic turbostatic carbon target to a desired substrate at low temperatures. The process may be carried our in high-vacuum conditions to prevent chemical reaction. This technique may be suitable for coating a variety of substrates such as temperature-sensitive polymers and metal alloys.

The high strength, resistance to breakdown or corrosion, and durability of a coated carbon surface ensures effective, long term functioning of coated particles in tissue modifying applications. The established biocompatibility of carbon coatings such as pyrolytic and vitreous carbon coatings makes the described particles particularly suitable for tissue modifying applications. The particulate substrates may be completely encased by a carbon surface. This results in a uniformly coated particle with no substrate exposure on the surface of the particle. Preferred carbon coatings may be in the range of fractions of thousandths of an inch, e.g., about one half of a thousands of an inch (0.0005 inches), on average, covering the surface of the particle substrate.

The particles, whether coated or uncoated, are preferably shaped and sized to provide enhanced passage through a hypodermic needle. In one embodiment the shape and size of the injected particles are varied to enhance the flow of the particles during injection. The particles may also be subjected to a cleaning, polishing and sieving process to remove contaminants, smooth the particle surface to a desired texture and to separate out particles of a size less than or greater than a desired size range. The particles may range in size from 10 microns to 1,000 microns in average, transverse cross-sectional dimension, and are preferably in the range from about 80 to 300 microns.

The biocompatible particles are delivered to the tissue site in a suitable biocompatible carrier. Any biocompatible carrier that can deliver the particles to a soft tissue site may be used in accordance with the present invention. A carrier may be a biologically compatible solution. Examples of suitable carriers include solutions containing glucan, collagen, saline, dextrans, glycerol, polyethylene glycol, corn oil or safflower, other polysaccharides or biocompatible polymers, methyl cellulose, agarose, or combinations thereof. In certain embodiments, a curable polymer such as PMMA, may be added to the carrier to provide additional stiffening characteristics. The viscosity of the carrier ranges between about 10 and 75,000 centipoise.

Solutions containing β-glucan and collagen are particularly suitable carriers for the present invention. β-glucan is a naturally occurring constituent of cell walls in essentially all living systems including plants, yeast, bacteria, and mammalian systems. Its effects and modulating actions on living systems have been reported by Abel et. al., “Stimulation of Human Monocyte B-glucan Receptors by Glucan Particles Induces Production of TNF-∂ and 1 L-B,” Int. J. Immunopharmacol., 14(8):1363-1373, 1992. β-glucan, when administered in experimental studies, elicits and augments host defense mechanisms including the steps required to promote healing, thereby stimulating the reparative processes in the host system. β-glucan is removed from tissue sites through macrophagic phagocytosis or by enzymatic destruction by serous enzymes. The destruction or removal of β-glucan, as well as its available viscosity and lubricous nature, make it a useful carrier for the particles in tissue modifying applications.

Aqueous solutions of β-glucan may be produced that have favorable physical characteristics as a carrier for particles in tissue modifying applications. The viscosity can vary from a thin liquid to a firm, self-supporting gel. Irrespective of viscosity, the β-glucan solution has excellent lubricity, thereby creating a particle-carrier composition which is easily administered by delivery to a predetermined body site through a small bore needle. Useful β-glucan compositions include β-D-glucans containing 4-0-linked-β-D-glycopyranosyl units and 3-0-linked-β-D-glycopyranosyl units, or 5-0-linked-β-D-glycopyranosyl units and 3-0-linked-β-D-glycopyranosyl units. The carrier may be of sufficient viscosity to assure that the particles remain suspended therein, for a sufficient time duration to accomplish the injection procedure.

Collagen is a naturally occurring protein that provides support to various parts of the human body, including the skin, joints, bone and ligaments. Injectable collagen manufactured by the McGhan Medical Corporation, Santa Barbara, Calif., and sold under the trade names ZYDERM and ZYPLAST, is derived from purified bovine collagen. The purification process results in a product similar to human collagen. Collagen solutions may be produced within a wide viscosity range to meet an individual patient's needs.

Another example of a suitable carrier material is a solution containing methyl cellulose or another linear unbranched polysaccharide. Further examples of appropriate carrier materials include agarose, hyaluronic acid, polyvinyl pyrrolidone or a hydrogel derivative thereof, dextran or a hydrogel derivative thereof, glycerol, polyethylene glycol, oil-based emulsions such as corn or safflower, or other polysaccharides or biocompatible organic polymers either singly or in combination with one or more of the above-referenced solutions.

The amount of particles in the modifier may be any amount that will provide a modifier that is flowable and injectable, and that will allow a desired amount of particles to be delivered to a tissue site. Amounts of particles in the soft tissue modifier can be in the range from about 5 to 85 percent by volume, more particularly from about 20 to 60 percent by volume, and most particularly from about 30 to 50 percent by volume.

In use, the modifier will typically be injected as a slurry, suspension, or emulsion, through a needle, into a tissue site. When deposited into a tissue site, the carrier may be carried away into the body and then be dispersed or destroyed. It is preferred that some of the particles are substantially immobile upon delivery to a tissue site for modification. Particles used for tissue modifying according to the invention may be sufficiently immobile to be used for substantially permanent tissue modifying applications. If the particles tend to move at all after delivery to a tissue site, the particles generally will do so only along the path of the needle that was used to inject them.

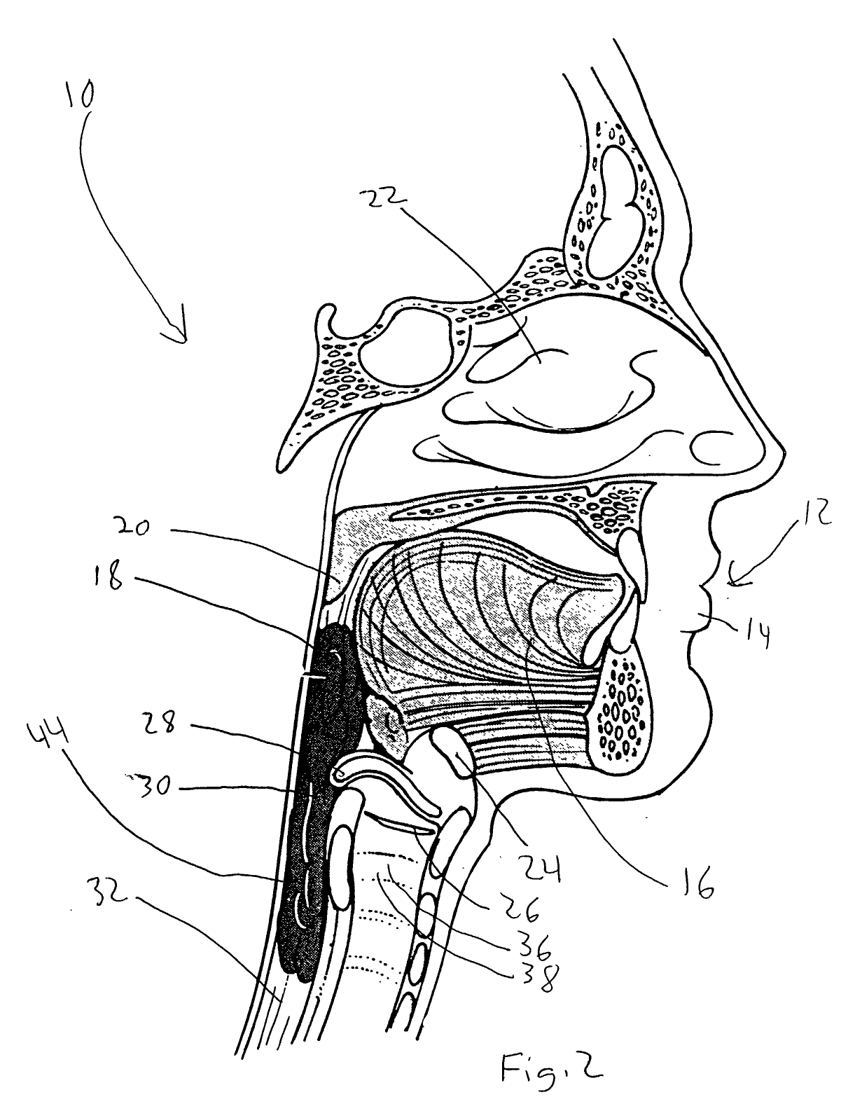

The modifier may be delivered to a tissue site using any instrument or apparatus that can be used to inject an amount of particles, preferably contained or suspended in a carrier, through the skin or mucosa, to a desired tissue site. Suitable instruments include hypodermic needles or other similar needle-like apparatuses, such as any small bore instrument, cannula, etc. (All of these types of instruments will be referred to collectively herein, for convenience, using the term “hypodermic needle” or “needle.”) The particular instrument used for delivery is not critical, provided that its components are compatible with the modifier. According to one example of a method of delivering particles for tissue modification, particles can be delivered using a hypodermic needle and a syringe, by inserting the hypodermic needle at, or in the vicinity of, a desired tissue site, followed by delivery of the particles to the tissue site. For example, the needle may be positioned within the mucosal, submucosal or muscular tissue layer of a tissue site. Referring to FIGS. 1 and 2, tissue sites suitable for injection include, but are not limited to, the mouth 12, tongue 16, lips 14, soft palate 20, pharynx 18, nasal passages 22, true vocal cords 36, false vocal cords 38, epiglottis 28, trachea 42, upper esophageal sphincter 30 and/or other suitable tissue sites of the swallowing system.

Once a needle is placed, particles may be slowly injected through the needle to the desired tissue site. In one embodiment, the particles are of a size that may be effectively deposited through a hypodermic needle or like instrument, and that will substantially remain at the tissue site where delivered. If the particles are too small, they may be engulfed by the body's white cells (phagocytes) and carried to distant organs or be carried away in the body's microvasculature system and travel until they reach a site of sufficient constriction to prevent further movement. On the other hand, particles are not so large that they cannot be effectively delivered using a hypodermic needle or the like. In one embodiment of the present invention, the average particle size may be from about 80 to 300 microns, because such sizes may allow injection through small bore instruments and are large enough to avoid migration of the particles from the injection site.

Optionally, prior to injecting the modifier, the tissue may be subjected to a hydrodissection process. During hydrodissection, a biocompatible liquid, such as a saline solution, is injected into a tissue site. The liquid injection provides an enlarged cavity within or between the tissue layer. The amount of injected liquid may range from 0.25 to 10 cc. After hydrodissection, the modifier may be injected into the enlarged cavity in the tissue site.

The use of particles in tissue modification, preferably injected by use of a needle and syringe or a like instrument, has advantages over the use of other tissue modification methods. For instance, delivery of particles using a needle and syringe allows very precise delivery of particles to a desired tissue site. Optionally, particular injection precision may accomplished with radiopaque embodiments of the present invention. Other advantages are that particles can be used at tissue sites where other types of modifiers either cannot be, or have not been used.

The amount of particles introduced to modify the tissue may be any amount sufficient to modify the desired site. The amount delivered may vary depending on factors such as the size of the particles, the extent of necessary modification, the tissue condition to be treated and other factors particular to specific patients. Such factors will be within the skill of an artisan of ordinary skill in the medical arts, and such an artisan will be able to understand what is a useful amount of particles for delivery to body tissue sites.

One characteristic of the modifier of the present invention is that it may be injected into tissue in incremental portions. In this manner, only the minimally necessary amount of modifier is added. This drastically reduces the possibility that the functionality of the tissue will be adversely effected by the modifier. In certain embodiments, the modifier may include detectable, for example, radiopaque particles. In these embodiments, the injected modifier may be viewed to determine the location of the particles within the tissue, and the overall effect of the modifiers. If, after a first injection, the dynamic response is not sufficiently altered, precise amounts of additional modifier may be injected into the tissue site. This incremental approach is particularly suitable for the modifier of the present invention because no invasive incision is required to inject the particles. The present invention may also be injected into two or more distinct tissue sites to modify the swallowing system as desired.

Another embodiment of the present invention provides a method of modifying swallowing system tissue wherein a biocompatible solution free of particles is injected into a tissue site. The method may be used to temporarily modify tissue to determine the source of a particular disorder, or to remedy a temporary disorder. Optionally, a modifier including biocompatible particles may be subsequently injected into the tissue site to provide permanent modification. In this manner, the modifier of the present embodiment may be used to diagnose a condition and experiment with possible solutions without permanently modifying tissue.

Certain particle-free biocompatible solutions, such as solutions containing β-glucan, may produce a fibroid response in the tissue site into which it was injected.

The result is that the modifier used in this embodiment provides a permanent modification of the tissue site by increasing the rigidity of the tissue without using particulate matter.

One of skill in the art will recognize that embodiments of the present invention may be used to modify the swallowing system to treat many conditions. In one embodiment, present invention may be used to reduce or prevent snoring by modifying the soft tissue of the swallowing system. Snoring is caused by vibrations of soft tissue in response to the passage of air through the swallowing system. Embodiments of the present invention may modify a soft tissue site in three primary ways. First, embodiments of the present invention may alter the mass of the soft tissue site. Second, embodiments of the present invention may alter the rigidity of the soft tissue site. Third, embodiments of the present invention may alter the geometry of the soft tissue site.

The mass of the soft tissue site may contribute to the dynamic response of the soft tissue site to the passage of air. For example, an end of the soft palate hangs down from the hard palate, and is generally responsible for vibrations that cause snoring. However, by increasing and/or relocating the mass of the soft palate, the dynamic response of the soft palate may be affected, without restricting the soft palate from closing off the nasal passages when a patient swallows.

The modifier may also alter the relative rigidity of the soft tissue site to modify dynamic response. For example, the modifier of the present invention may be injected into the soft palate to increase tissue rigidity. This may allow the soft palate to resist deflections caused by the passage of air. Tissue modifiers of the present invention may be precisely injected into the soft tissue site to provide the desired result. Further, these embodiments may possess viscosities that are capable of stiffening and dampening the soft tissue site. In one embodiment, a curable or hardenable polymer may be added to further stiffen the soft tissue site. For example, PMMA may be added to the present modifier to provide increased rigidity. Alternatively, a flexible, injectable polymeric substance may be added to the modifier to provide increased flexibility of the modifier. Again, because the present invention may be injected in precise amounts and at precise locations, the patient is less likely to lose functionality of the soft tissue.

The overall shape of the soft tissue may also modify dynamic response to the passage of air. For example, the passage of air underneath the soft palate may act to lift the soft palate towards the nasal passages, resulting in the snoring response. However, the present invention may be used to alter the shape of the soft palate or other tissue sites to reduce or prevent this lifting action.

Additionally, embodiments of the present invention may be used to treat sleep apnea by altering the shape of the soft tissue of the tongue and/or throat to reduce or prevent closure of the airway. Stagnant pockets or cavities in the mouth and pharynx caused by the removal of cysts or tumors may be filled or modified. The nasal passages may be altered to reduce or prevent airway obstruction. Speech impediments may be treated by affecting the shape of the mouth, tongue or jaw.

In one embodiment, the modifier of the present invention may be used to treat patients having difficulty swallowing. Difficulty swallowing may be caused by many tissue conditions, including reduced and/or disorganized range of tongue motion, reduced labial closure, tongue thrust, altered tongue contour, reduced velopharyngeal closure, laryngopharynx dysfunction, lip configuration, soft palate and uvular dimensions and damaged vocal folds.

The modifier of the present invention may be injected at, or in the vicinity of one or more of these tissue sites to modify, bulk or augment damaged or deformed tissue. For example, the modifier may be injected into the lips, tongue or soft palate to provide the correct contours or range of motion required for proper swallowing. In another example, the modifier may be injected into the true or false vocal folds to augment damaged tissue and improve swallowing. In a further example, the modifier may be injected at or in the vicinity of the upper esophageal sphincter to enhance the closing function of this tissue site.

This invention is not to be taken as limited to all of the above described details thereof as modifications and variations thereof may be made without departing from the spirit or scope of the invention.

What is claimed is:

1. A method of modifying the swallowing system of a patient comprising injecting a modifier comprising biocompatible particles and a biocompatible carrier into a tissue site of the patient.

2. The method of claim 1 wherein the biocompatible particles comprise ceramic particles.

3. The method of claim 1 wherein the biocompatible particles comprise a polymeric material.

4. The method of claim 1 wherein the biocompatible particles have an exposed surface of carbon.

5. The method of claim 4 wherein the injecting step further comprises injecting the modifier at a tongue, lip, soft palate, pharynx, nasal passages, epiglottis, trachea, aryepiglotic folds, upper esophageal sphincter, cricoid cartilage, true vocal cords or false vocal cords of the patient.

6. The method of claim 4 wherein the particles comprise graphite.

7. The method of claim 4 wherein the particles comprise isotropic pyrolytic carbon.

8. The method of claim 4 wherein the exposed surface of carbon is an exposed surface of isotropic pyrolytic carbon.

9. The method of claim 4 wherein the particles comprise a particulate substrate coated with isotropic pyrolytic carbon.

10. The method of claim 9 wherein the biocompatible particles comprise a graphite particulate substrate coated with isotropic pyrolytic carbon.

11. The method of claim 9 wherein the biocompatible particles comprise a polymeric substrate coated with isotropic pyrolytic carbon.

12. The method of claim 1 wherein the biocompatible particles have a transverse cross-section dimension between about 10 and 1000 microns.

13. The method of claim 1 wherein the biocompatible particles have a transverse cross-section dimension between about 80 and 300 microns.

14. The method of claim 1 wherein the biocompatible carrier is a solution.

15. The method of claim 14 wherein the biocompatible carrier includes polymethylmethacrylate, a polysaccharide or collagen.

16. The method of claim 14 wherein the biocompatible carrier includes β-glucan.

17. The method of claim 1 wherein the modifier has a viscosity of between about 10 and 75,000 centipoise.

18. The method of claim 1 wherein the modifier comprises about 5 to 85 v/v percent biocompatible particles.

19. The method of claim 1 wherein the modifier comprises about 20 to 60 v/v percent biocompatible particles.

20. The method of claim 1 wherein the modifier comprises about 30 to 50 v/v percent biocompatible particles.

21. The method of claim 1 further comprising detecting the biocompatible particles.

22. The method of claim 21 wherein the detecting step occurs during the injecting step.

23. The method of claim 1 wherein the injecting step further comprises injecting the modifier at a tongue, lip, soft palate, pharynx, nasal passages, epiglottis, trachea, aryepiglotic folds, upper esophageal sphincter or cricoid cartilage of the patient.

24. The method of claim 1 wherein the injecting step further comprises injecting the modifier into the soft palate of the patient.

25. The method of claim 1 wherein the injecting step comprises injecting the modifier into a subcutaneous, mucosal, submucosal, or muscle layer of the tissue site of the patient.

26. The method of claim 1 further comprising injecting a biocompatible liquid into the tissue site to provide an internal space for injecting the modifier.

27. The method of claim 1 wherein the injecting step further comprises injecting the modifier into a tissue site of a patient to treat snoring, voice conditions, swallowing disorders, speech impediments, stagnant pockets, sleep apnea, soft tissue cavities or aspiration.

28. A method of modifying a tissue site of the swallowing system of a patient comprising injecting a modifier consisting of a biocompatible solution into the tissue site of the patient.

29. The method of claim 28 wherein the modifier temporarily modifies the tissue site.

30. The method of claim 28 wherein the modifier produces a fibroid response at the tissue site to substantially permanently modify the tissue site.

31. The method of claim 30 wherein the biocompatible carrier is a solution containing β-glucan.

32. The method of claim 28 further comprising subsequently injecting a modifier comprising biocompatible particles and a biocompatible carrier into the tissue site of the patient to substantially permanently modify the tissue site.

33. The method of claim 28 wherein the injecting step further comprises injecting the modifier into a tongue, lip, soft palate, pharynx, nasal passages, true vocal cords, false vocal cords, epiglottis, trachea, aryepiglotic folds, upper esophageal sphincter or cricoid cartilage of the patient.

34. The method of claim 28 wherein the injecting step further comprises injecting the modifier into the soft palate of the patient.

35. A method of treating snoring of a patient comprising injecting an effective amount of a modifier comprising biocompatible particles and a biocompatible carrier into a soft tissue site of the swallowing system of the patient to modify the dynamic response of the tissue site to the passage of air through the swallowing.

36. The method of claim 35 wherein the modifier increases the mass of the soft tissue site to modify the dynamic response.

37. The method of claim 35 wherein the modifier alters the rigidity of the soft tissue site to modify the dynamic response.

38. The method of claim 35 wherein the modifier alters the geometry of the soft tissue site to modify the dynamic response.

39. The method of claim 35 wherein the tissue site comprises a nasal passages, tongue, soft palate, pharynx, lip, esophagus, true vocal cords, false vocal cords, epiglottis, trachea, aryepiglotic folds, or upper esophageal sphincter of the patient.

40. The method of claim 35 wherein the tissue site comprises the soft palate of the patient.

41. The method of claim 35 comprising injecting the modifier at a second tissue site.

42. The method of claim 35 further comprising detecting the biocompatible particles during the injecting step.

43. The method of claim 35 further comprising detecting the biocompatible particles after the injecting step.

44. The method of claim 35 further comprising injecting an additional amount of modifier into the tissue site to further modify the dynamic response of the soft tissue site to the passage of air through the upper airway.
